(19) United States
(12) Patent Application Publication
(10) Pub. No.: US 2008/0208202 A1
(43) Pub. Date: Aug. 28, 2008

(54) ADAPTABLE TOOL REMOVAL DEVICE AND METHOD

(76) Inventor: Jeffrey Williams, Plainville, MA (US)

Correspondence Address: CARR LLP (IST), 670 FOUNDERS SQUARE, 900 JACKSON STREET, DALLAS, TX, 75202, US

(21) Appl. No.: 12/018,913

(22) Filed: Jan. 24, 2008

Related U.S. Application Data
(60) Provisional application No. 60/886,589, filed on Jan. 25, 2007.

(51) Int. Cl. A61B 17/58; A61B 17/56

(52) U.S. Cl. 606/100; 606/86 R

(57) ABSTRACT

The present invention may provide for an adaptable medical tool removal device and method for removing medical instruments seized in vivo. The removal tool may comprise a clamping mechanism. The clamping mechanism may comprise a locking member, a pivotal clamp member, and a connector clamp member. The pivotal clamp member may be pivotally attached to the connector clamp member. The pivotal clamp member may pivot between an opened and a closed position. The locking member may lock the pivotal clamp member in a closed position around a tool adaptor device. The tool adaptor device may attach to a seized medical instrument. The removal tool may further comprise a sliding weight able to impart an impact force to the seized medical instrument through the tool adaptor device. An embodiment of the tool adaptor device may comprise hooks to connect to a t-shaped handle of a seized medical instrument.

### CROSS-REFERENCED APPLICATIONS

This application relates to, and claims the benefit of the filing date of, co-pending U.S. provisional patent application Ser. No. 60/886,589 entitled ADAPTABLE TOOL REMOVAL DEVICE AND METHOD, filed Jan. 25, 2007, the entire contents of which are incorporated herein by reference for all purposes.

### BACKGROUND OF THE INVENTION

1. Field of the Invention

The present invention relates to medical instruments and, more particularly, to medical tool removal instruments.

2. Description of the Related Art

During the course of invasive medical and surgical procedures, medical tools and instruments may become trapped or caught by interior surfaces of a patient's body (e.g., between bony surfaces, among others). Current methods of removing a seized medical instrument involve specifically designed removal tools configured to be attached to the particular seized instrument and other handheld devices such as hammers. Depending upon the procedure performed, an operating staff may have to maintain an inventory of medical instruments and a corresponding inventory of specific removal tools for each of those instruments. In addition, a surgeon may have to release the seized instrument in order to attach the removal tool or to apply a force via a separate hammer. An adaptable medical removal tool is needed that is readily attachable to a variety of seized medical instruments, and operable while retaining control of the seized instrument.

### SUMMARY OF THE INVENTION

The present invention provides a medical removal tool that may comprise a first member, a second member, and a lock member. The second member may be pivotally coupled to the first member. The second member may pivot between a first position and a second position. The lock member may restrain the second member in one of the first position and the second position. The shaft may comprise a mass member and an end member. A mass member may be slidably attached to the shaft. When the mass member impacts the end member, a force may be transmitted to the first and second members. However, it should be understood that the invention may have uses in addition to the removal of medical instruments.

### BRIEF DESCRIPTION OF THE DRAWINGS

For a more complete understanding of the present invention and the advantages thereof, reference is now made to the following Detailed Description taken in conjunction with the accompanying drawings, in which:

FIG. 1 illustrates a side view of a removal tool in accordance with an embodiment of the present invention;

FIG. 2 illustrates an enlarged detail view of a clamping mechanism of FIG. 1;

FIG. 3A illustrates a top perspective view of a retainer of FIG. 2;

FIG. 3B illustrates a cross-sectional view of the retainer of FIG. 3A taken along the line 3B-3B;

FIG. 4A illustrates a bottom perspective view of a locking member of FIG. 2;

FIG. 4B illustrates a cross-sectional view of the locking member of FIG. 4A taken along the line 4B-4B;

FIG. 5A illustrates a top perspective view of a connector clamp member of FIG. 2;

FIG. 5B illustrates a top view of the connector clamp member of FIG. 5A;

FIG. 5C illustrates a cross-sectional view of the connector clamp member of FIG. 5A taken along the line 5C-5C;

FIG. 5D illustrates a front view of the connector clamp member of FIG. 5A;

FIG. 6A illustrates a top perspective view of a pivotal clamp member of FIG. 2;

FIG. 6B illustrates a top view of the pivotal clamp member of FIG. 6A;

FIG. 7 illustrates a top view of a resilient member of FIG. 2;

FIG. 8 illustrates a cross-sectional detail view of a distal portion of the removal tool of FIG. 1,

FIG. 9 illustrates a partial assembly view of the connector clamp member, resilient members, and pivotal clamp member of FIG. 2;

FIG. 10 illustrates a lower assembly view of the removal tool of FIG. 1 in an unlocked configuration;

FIG. 11A illustrates a lower assembly view of the removal tool of FIG. 1 in a locked configuration;

FIG. 11B illustrates a top cross-sectional view of the tool adaptor interface of the removal tool of FIG. 1 in a locked configuration;

FIG. 12 illustrates an embodiment of a tool adaptor device configured to couple with the removal tool of FIG. 1;

FIG. 13 illustrates another embodiment of a tool adaptor device configured to attach to the removal tool of FIG. 1;

FIG. 14A illustrates an enlarged side partial cross-sectional view of another embodiment of a clamping mechanism detailing a locking member in an unlocked position;

FIG. 14B illustrates the view of FIG. 14A in a locked position;

FIG. 14C illustrates a top perspective view of an embodiment of a locking member of FIG. 14A; and

FIG. 15 illustrates another embodiment of a clamping mechanism of the removal tool of FIG. 1.

### DETAILED DESCRIPTION

In the following discussion, numerous specific details are set forth to provide a thorough understanding of the present invention. However, those skilled in the art will appreciate that the present invention may be practiced without such specific details. In other instances, well-known elements have been illustrated in schematic or block diagram form in order not to obscure the present invention in unnecessary detail. Additionally, for the most part, minor details have been omitted inasmuch as such details are not considered necessary to obtain a complete understanding of the present invention, and are considered to be within the understanding of persons of ordinary skill in the relevant art.

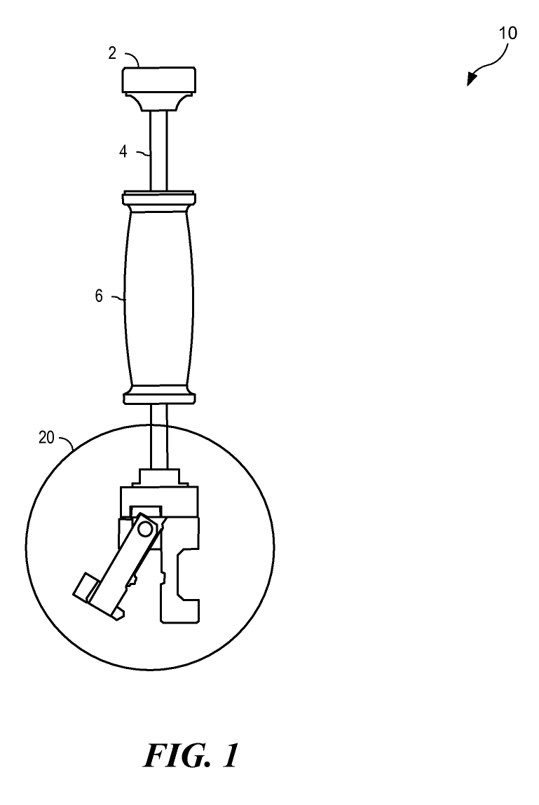

Turning now to FIG. 1, the reference numeral 10 generally indicates an illustrative embodiment of a removal tool 10 of the present invention. The removal tool 10 may comprise a handle 2, a slide shaft 4, a slide weight 6, and a clamping mechanism 20. The slide weight 6 may be slidably attached to the slide shaft 4 and configured to travel along an axis of the slide shaft 4. The handle 2 may be securely attached to a proximate end of the slide shaft 4. Examples of securely attaching the first impact member 2 to the proximate end of the slide shaft 4 may comprise threadably securing, welding, gluing, and forming about the proximate end, among others. Alternatively, the handle 2 may be integrally formed along with the slide shaft 4, for example, through machining, forging, casting, and molding, among others.

### Clamping Mechanism

The clamping mechanism 20 may be securely attached to a distal end of the slide shaft 4. Examples of securely attaching the clamping mechanism 20 to the slide shaft 4 may comprise threadably securing, welding, gluing, and forming of some components of the clamping mechanism 20 around the distal end of the slide shaft 4. Alternatively, some components of the clamping mechanism 20 may be integrally formed along with the slide shaft 4, for example, through machining, forging, casting, and molding, among others.

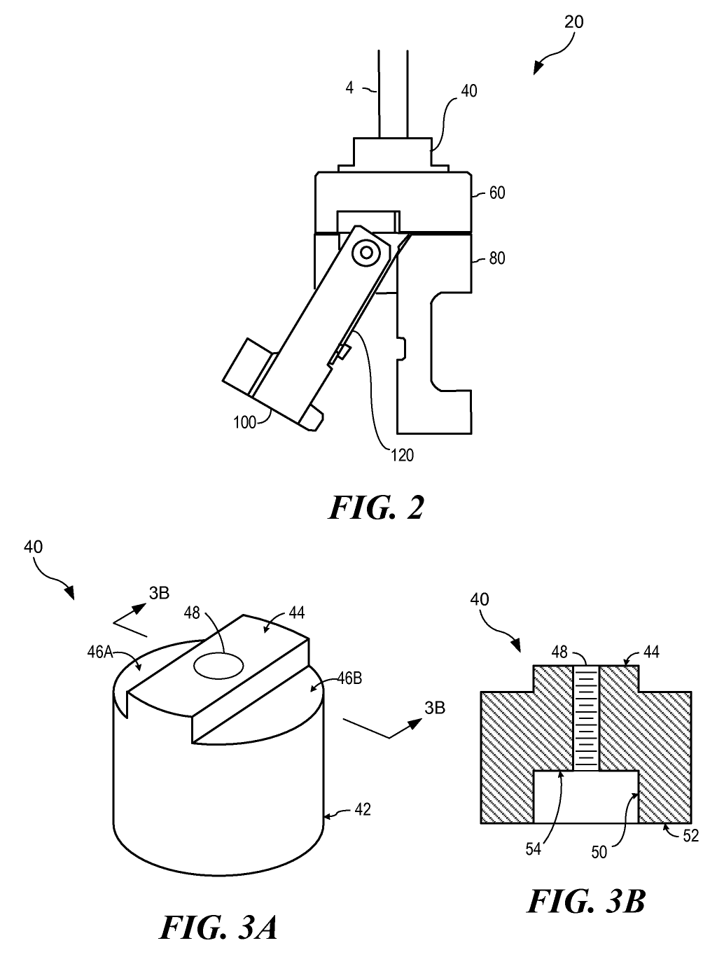

Turning now to FIG. 2, the clamping mechanism 20 may comprise a retainer 40, a locking member 60, a connector clamp member 80, a pivotal clamp member 100, and a resilient member 120. The retainer 40 and the connector clamp member 80 may be securely attached to the slide shaft 4. The locking member 60 may be rotatably coupled to the connector clamp member 80 and slidably interfaced with the pivotal clamp member 100. The pivotal clamp member 100 may be pivotally or rotatably coupled to the connector clamp member 80. The resilient member 120 may be attached to the connector clamp member 80 and the pivotal clamp member 100.

### Retainer

Turning now to FIG. 3A, the retainer 40 may comprise an outer retainer circumference 42, a retainer impact surface 44, tool recesses 46A, 46B, and a retainer threaded interface 48. The tool recesses 46A, 46B at the proximate end of the retainer 40 may be shown as substantially symmetrical about the center axis of the retainer 40. The tool recesses 46A, 46B may form a tool interface comprising a raised plateau including the retainer impact surface 44. During assembly, a tool (not shown) may slidingly engage this tool interface so as to impart a rotating torque to the retainer 40. Although two tool recesses 46A, 46B may be shown, the retainer 40 may not be limited to this configuration. Any tool interface may be integrated with the retainer 40, including, but not limited to, a hex-shaped interface, one or more recesses located about the circumference 42, and one or more grooves cut into the retainer impact surface 44.

Turning now to FIG. 3B, the retainer 40 may further comprise an inner retainer circumference 50, a retainer distal surface 52, and an inner retainer surface 54. The inner retainer circumference 50 and the inner retainer surface 54 substantially form a bottomed cylindrical cavity within the retainer 40. The inner retainer circumference 50 and the inner retainer surface 54 may be approximately orthogonal to each other. The retainer distal surface 52 may be substantially planar and orthogonal to a central axis of the retainer 40, and substantially parallel to the retainer impact surface 44. The retainer threaded interface 48 may extend through the thickness of the retainer 40, from the retainer impact surface 44 to the inner retainer surface 54.

### Locking Member

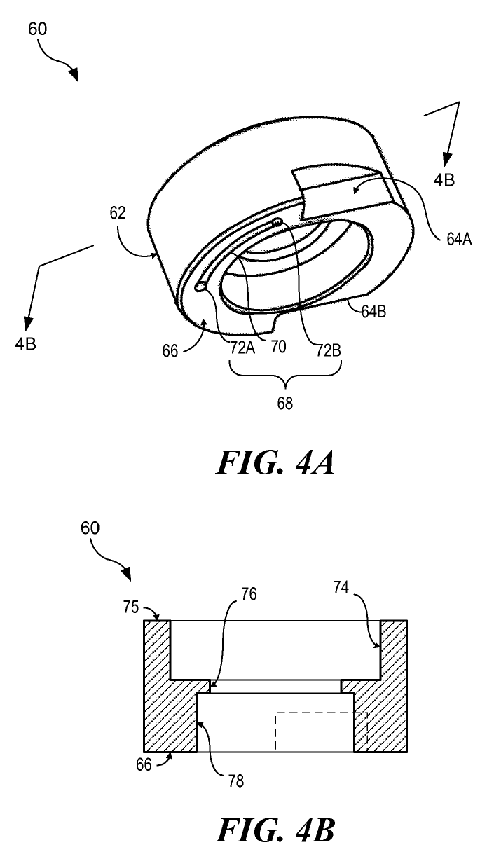

Turning now to FIG. 4A, the locking member 60 may comprise an outer locking circumference 62, cam recesses 64A, 64B, locking distal surface 66, and limiting interface 68. The outer locking circumference 62 and the locking distal surface 66 may comprise one or more cam recesses 64A, 64B. In the example of an embodiment of the present invention shown in FIG. 4A, the one or more cam recesses 64A, 64B may be symmetrical about a plane passing through a central axis of the locking member 60. As seen in this figure, a relatively straight edged cavity may define the cam recesses 64A, 64B. However, many forms and configurations without limit may be used to provide clearance for a cam surface. As an example, cam recesses may comprise semi-circular recesses, grooves, and slots formed in either or both of the outer locking circumference 62 and the locking distal surface 66.

The locking member 60 may comprise a limiting interface 68. The limiting interface 68 may constrain the rotation of an assembled locking member 60 to within a desired angular range. An example of a desired angular range may be the range including 0°-90°. An example of the limiting interface 68 may comprise a limit track 70 and limit detents 72A, 72B located at the ends of the limit track 70. The limit track 70 and limit detents 72A, 72B may interact with a ball and spring assembly (not shown in this view) to restrict the rotation of the locking member 60. The limit detents 72A, 72B may provide a slight feedback when engaging the ball of the ball and spring assembly, indicating that the locking member 60 has reached a limiting point in rotation. For example, a first limit detent 72A may be configured to coincide with an unlocked position of the locking member 60, while a second limit detent 72B may be configured to coincide with a locked position of the locking member 60. In addition, the engagement between the ball and the limit detents 72A, 72B may provide a retention force for maintaining an assembled locking member 60 at either end of rotation. The retention force may inhibit or prevent inadvertent or unintentional rotation of the locking member 60, possibly reducing the chance that the locking member 60 may become unlocked during a tool removal procedure. A limit track 70 and limit detents 72A, 72B may be shown as an example of a limiting interface 68. However, any limiting interface may be used in an embodiment of the present invention. Another example may include one or more protrusions on a surface of a component such as the locking member 60, engaging with corresponding slots on an abutting surface of a component such as the connector clamp member 80 (FIG. 2), among others.

Turning now to FIG. 4B, the locking member 60 may comprise a first inner circumference 74, a second inner circumference 76, and a third inner circumference 78. The first inner circumference 74 may be adjacent to a locking proximate surface 75. The third inner circumference 78 may be adjacent to a locking distal surface 66. The second inner circumference 76 may define a protrusion located between the first inner circumference 74 and the third inner circumference 78. The first inner circumference 74 in this illustrative embodiment may be shown as larger or smaller than the third inner circumference 78. However, the first inner circumference 74 may be equal to the third inner circumference 78.

### Connector Clamp Member

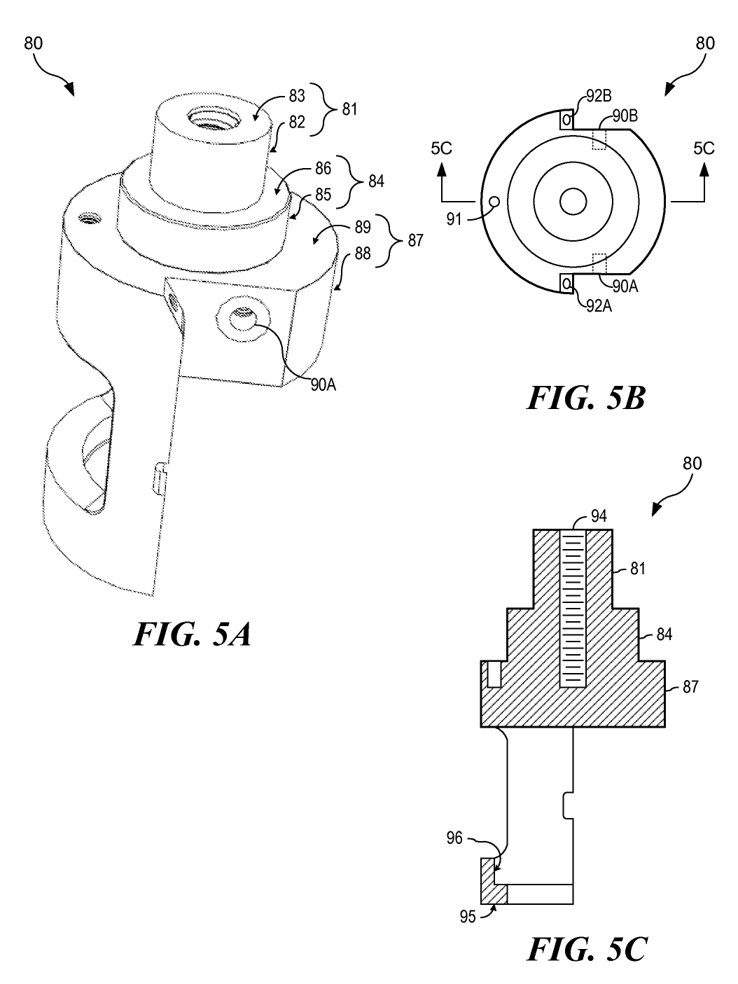

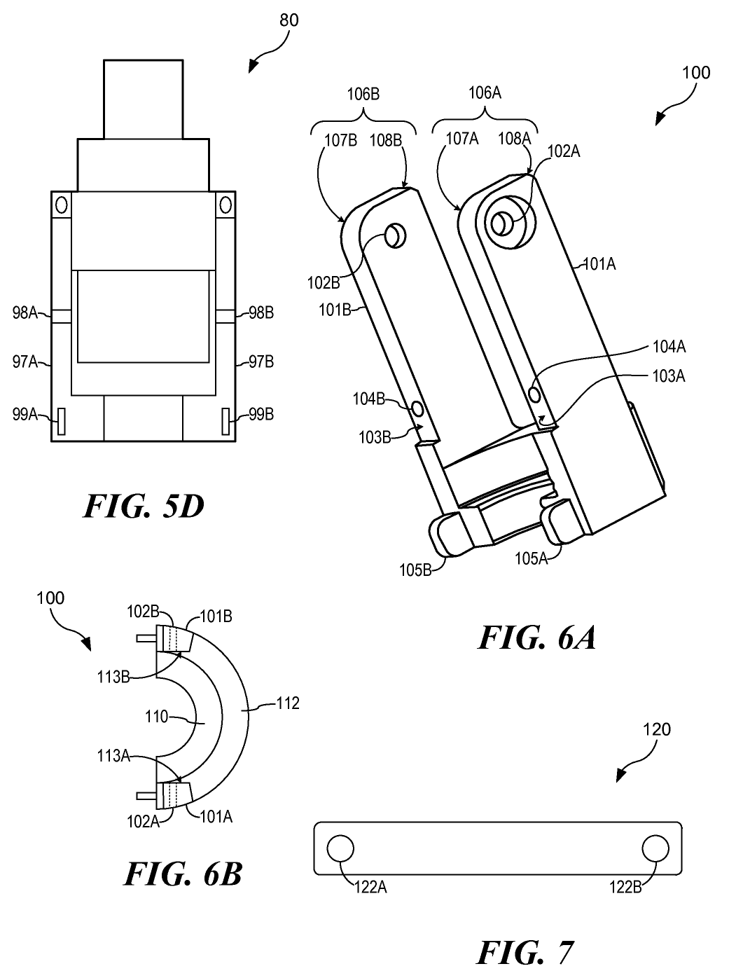

Turning now to FIG. 5A, the connector clamp member 80 may comprise an extension 81, a locking member interface 84, and a clamp base 87. The extension 81 may be located on a proximate end of the connector clamp member 80 and comprise an extension circumference 82 and an extension surface 83. The locking member interface 84 may be adjacent to the extension 81 and comprise a locking interface circumference 85 and a locking interface surface 86. The clamp base 87 may be located adjacent to the locking member interface 84 and comprise a clamp circumference 88 and a clamp surface 89. One or more pivot points 90A, 90B (only 90A may be seen in this view) may be located on a side of the clamp base 87.

Turning to FIG. 5B, the connector clamp member 80 may comprise one or more pivot points 90A, 90B (see also FIG. 5A), limiting device interface mount 91, and proximate resilient member mounts 92A, 92B. The two (in this example) pivot points 90A, 90B, may be coincident with each other across a width of the connector clamp member 80. In some embodiments, the two pivot points 90A, 90B may be replace by a single through hole. In this embodiment, portions of the clamp circumference 88 may be planar so as to provide substantially parallel pivot surfaces around the pivot points 90A, 90B. The pivot surfaces may be substantially orthogonal to a central axis of the pivot points 90A, 90B.

The limiting device interface mount 91 may accommodate a ball and spring assembly (not shown in this view) for engaging with a limiting interface 68 (FIG. 4A). The proximate resilient member mounts 92A, 92B, may each provide a mounting location for one end of a resilient member 120 (FIG. 2) detailed later. The proximate resilient member mounts 92A, 92B may be at an angle to the clamp surface 89 (FIG. 5A).

Turning now to FIG. 5C, the connector clamp member 80 may comprise a clamp threaded interface 94, a tool adaptor plate 95, and a tool adaptor wall 96. The clamp threaded interface 94 may extend along a central axis of the connector clamp member 80 through the extension 81, the locking member interface 84, and a portion of the clamp base 87, for example. The tool adaptor plate 95 may be configured to abut and retain an end portion of a tool adaptor device (not shown) detailed later. The tool adaptor wall 96 may strengthen and surround an outer perimeter of a portion of the tool adaptor plate 95.

Turning now to FIG. 5D, the connector clamp member 80 may comprise one or more clamp support arms 97A, 97B. Two clamp support arms 97A, 97B, may be shown in this illustrative embodiment, but an embodiment of the present invention may not be limited to this configuration. The support arms 97A, 97B of the connector clamp member 80 may comprise distal resilient mount recesses 98A, 98B, and alignment orifices 99A, 99B. The distal resilient mount recesses 98A, 98B may provide clearance for fastening devices used to attach a distal end of the resilient members 120 (FIG. 2), for example. In some embodiments, the clearance may be needed when a clamping mechanism 20 (FIG. 2) is in a closed configuration. Similarly, the alignment orifices 99A, 99B may provide alignment and strengthen the clamping mechanism 20 in the closed position. The alignment orifices 99A, 99B may accommodate alignment protrusions (detailed later) provided on the pivotal support arms of the pivotal clamp member 100 (detailed later).

### Pivotal Clamp Member

Turning now to FIG. 6A, the pivotal clamp member 100 may comprise one or more pivotal support arms 101A, 101B. Each of the pivotal support arms 101A, 101B may comprise corresponding pivotal connector points 102A, 102B, resilient member recesses 103A, 103B, distal resilient member mounts 104A, 104B, and alignment protrusions 105A, 105B. The alignment protrusions 105A, 105B may be respectively located at a distal end of their respective pivotal support arms 101A, 101B. The number of pivotal support arms 101A, 101B, may correspond to the number of clamp support arms 97A, 97B (FIG. 5D). However, the number of pivotal support arms 101A, 101B, may not be limited to being equal to the number of clamp support arms 97A, 97B.

Each of the pivotal support arms 101A, 101B may comprise a corresponding cam surface 106A, 106B located at a proximal end of the pivotal support arms 101A, 101B. The cam surfaces 106A, 106B may respectively comprise a relatively arculate portion 107A, 107B, centered about the pivotal connector points 102A, 102B. In addition, the cam surfaces 106A, 106B may respectively comprise a cam lobe 108A, 108B extending radially beyond the surface defined by the arculate portions 107A, 107B. The pivotal connector points 102A, 102B may be coincident with each other. Consequently, rotation of the pivotal support arms 101A, 101B about the pivotal connector points 102A, 102B, may result in the rotation of respective cam lobes 108A, 108B.

Turning now to FIG. 6B, the pivotal clamp member 100 may comprise a tool adaptor plate 110, tool adaptor wall 112, and interior sections 113A, 113B. The tool adaptor plate 110 may be configured to abut and retain a portion of a tool adaptor device (not shown) detailed later. The tool adaptor wall 112 may strengthen and surround the outer perimeter of a portion of the tool adaptor plate 110. The pivotal support arms 101A, 101B may comprise substantially planar interior sections 113A, 113B, opposing one another. The interior sections 113A, 113B may be substantially parallel to each other and orthogonal to respective pivotal connector points 102A, 102B. The distal end of the pivotal support arms 101A, 101B may be integrally attached to a tool adaptor plate 110.

### Resilient Member

Turning now to FIG. 7, an illustrative embodiment of the resilient member 120 may comprise mounting orifices 122A, 122B. One of the mounting orifices 122A, 122B may be located at a proximate end of the resilient member 120. The other of the mounting orifices 122A, 122B may be located at a distal end of the resilient member 120. The resilient member 120 of an embodiment of present invention may be in the form of a relatively flat, leaf spring, but the form of the resilient member 120 may not be limited to this configuration. The resilient member 120 may be formed of a thin plate material such as stainless-steel, spring steal, engineered composite materials, among others. The mounting orifices 122A, 122B may be used for fastening devices (not shown in this view) to secure the resilient member 120 to the connector clamp member 80 and the pivotal clamp member 100 (see FIG. 2).

### Assembly

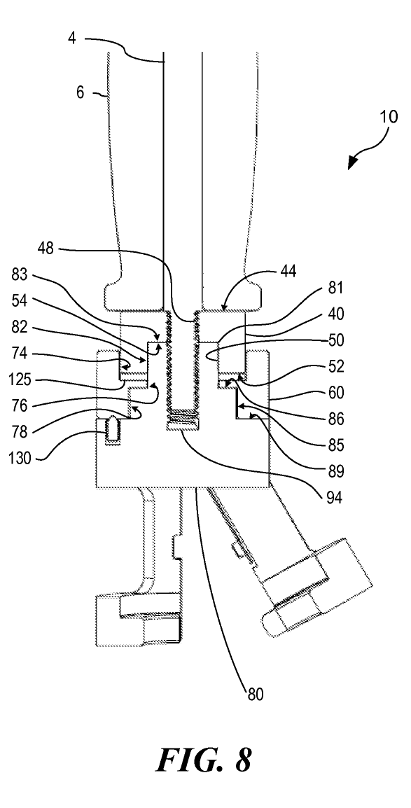

Turning now to FIG. 8, the slide weight 6 may be shown as slidably coupled with the slide shaft 4. In the figure, the slide weight 6 may be shown at a distal end of travel along the slide shaft 4, abutting the retainer impact surface 44 of the retainer 40. The retainer 40 may be threadably attached to a distal end of the slide shaft 4 via the retainer threaded interface 48. However, the attachment of the retainer 40 may not be limited to this example. The retainer 40 may be integrally formed with the slide shaft 4, for example, through machining. Additionally, the retainer 40 may be attached via welding, soldering, mechanical fasteners, set screws, retainers, keyways, lock rings, and chemical adhesive, among others.

The retainer 40 may be adjacent to a washer 125. The washer 125 may be a wave washer for example. The washer 125 may be configured to substantially correspond to the retainer distal surface 52. An inner circumference of the washer 125 may be substantially equal to the inner retainer circumference 50. The locking member 60 may be adjacent to an opposing surface of washer 125 relative to the retainer 40. The second inner circumference 76 of the locking member 60 may be substantially equal to the inner circumference of the washer 125 and the inner retainer circumference 50. The inner circumference of the washer 125, the inner retainer circumference 50, and the second inner circumference 76 of the locking member 60, may all be located by the extension circumference 82 of the extension 81 of the connector clamp member 80. Additionally, a distal portion of the retainer 40 may slidably fit within a portion of a cylindrical cavity defined by the first inner circumference 74 of the locking member 60 and the protrusion formed by the second inner circumference 76. The locking member 60 may rotate relative to the retainer 40.

The connector clamp member 80 may be adjacent to the locking member 60. The connector clamp member 80 may be threadably attached to the distal end of the slide shaft 4 via the clamp threaded interface 94. The extension 81 portion of the connector clamp member 80 may be positioned adjacent to the retainer 40 such that the inner retainer surface 54 abuts the extension surface 83 of the extension 81. When the inner retainer surface 54 abuts the extension surface 83, the connector clamp member 80 may be further rotated relative to the retainer 40. This may result in the retainer 40 functioning as a type of threadable locking device in order to prevent the accidental and/or inadvertent loosening of the threaded connections of one or both of the retainer 40 and the connector clamp member 80.

When fully assembled, the distance between the retainer distal surface 52 and the locking interface surface 86 of the connector clamp member 80 may be slightly larger than the combined thickness of the washer 125 and the protrusion of the locking member 60 resulting in the second inner circumference 76. Altering the distance between the retainer distal surface 52 and the locking interface surface 86 may vary the amount of rotational friction provided against the rotation of the locking member 60 relative to the retainer 40 and the connector clamp member 80. Alternatively or in addition to this method, structural configurations may be used to alter or vary the amount of rotational friction provided for the locking member 60. For example, the washer 125 may comprise a wave washer to resiliently provide an appropriate force against the rotation of the locking member 60 and to inhibit or prevent the rattling of the components of the assembled clamping mechanism 20. The wave washer may provide a force to bias the locking member 60 against the connector clamp member 80.

The locking distal surface 66 may slidingly abut the clamp surface 89. The clamp surface 89 may comprise a ball and spring mechanism 130. The ball and spring mechanism 130 may engage the limiting interface 68 (FIG. 4A). The ball may roll along the limit track 70 until an unlocked or a locked position of the locking member 60 is reached. At either or both of these points, the ball may engage a limit detent 72A, 72B, positioned at either end of the limit track 70. The reaction of the locking member 60 during the engagement of the ball with the limit detents 72A, 72B may provide feedback to an operator of the removal tool 10 to indicate a limit to further rotation of the locking member 60. The engagement of the ball with the limit detents 72A, 72B may also provide a securing force to inhibit or prevent the unintentional rotation of the locking member 60 due to an accidental striking of the locking member 60, or other interactions with the working environment, among others. Therefore, the chance of the locking member 60 becoming accidentally unlocked during a medical procedure may be reduced.

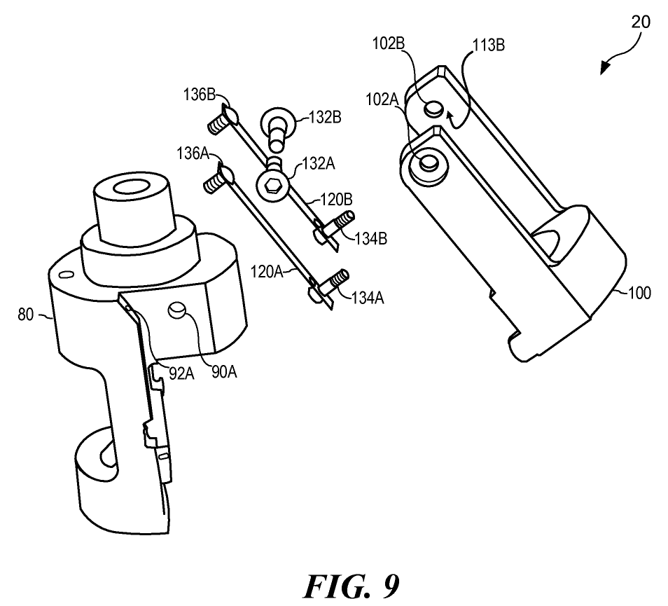

Turning now to FIG. 9, the pivotal clamp member 100 may be pivotally attached to the connector clamp member 80 at the pivotal connector points 102A, 102B. Two mounting bolts 132A, 132B, for example such as shoulder bolts, among others, may be inserted through the pivotal connector points 102A, 102B and threaded into the pivot points 90A, 90B (FIG. 5B). Although, two mounting bolts 132A, 132B may be described in this illustrative embodiment of the present invention, many other methods may exist to pivotally secure the pivotal clamp member 100 to the connector clamp member 80. For example, a single shaft threadably connected to fastening nuts on each end, various bushings, bearings, anti-friction devices, pressed pins, and rivets, among others, may be used in place of or in addition to the mounting bolts 132A, 132B.

The pivotal clamp member 100 may abut the connector clamp member 80 via the interior sections 113A, 113B (FIG. 6B) along the substantially planar areas surrounding the pivot points 90A, 90B (FIG. 5B). The use of substantially planar sections orthogonal to an axis coincident to the pivot points 90A, 90B, and the pivotal connector points 102A, 102B, may provide a consistent abutment surface for the relative rotation of the pivotal clamp member 100 and the connector clamp member 80.

In certain embodiments, the proximal ends of the resilient members 120A, 120B may be secured to the proximate resilient member mounts 92A, 92B (FIG. 5B) of the connector clamp member 80 via fasteners 136A, 136B. However, the attachment of the resilient members 120A, 120B may not be limited to this embodiment. The proximal ends of the resilient members 120A, 120B, may be coupled to the connector clamp member 80 via insertable slots, rivets, adhesive, soldering, and welding, among others. The distal ends of the resilient members 120A, 120B may be mounted to the distal resilient member mounts 104A, 104B (FIG. 6A), via fasteners 134A, 134B. As with the proximal mounting of the resilient members 120A, 120B, the distal ends of the resilient members 120A, 120B may be mounted to the pivotal clamp member 100 via insertable slots, rivets, adhesive, soldering, and welding, among others.

### Locked and Unlocked Configurations

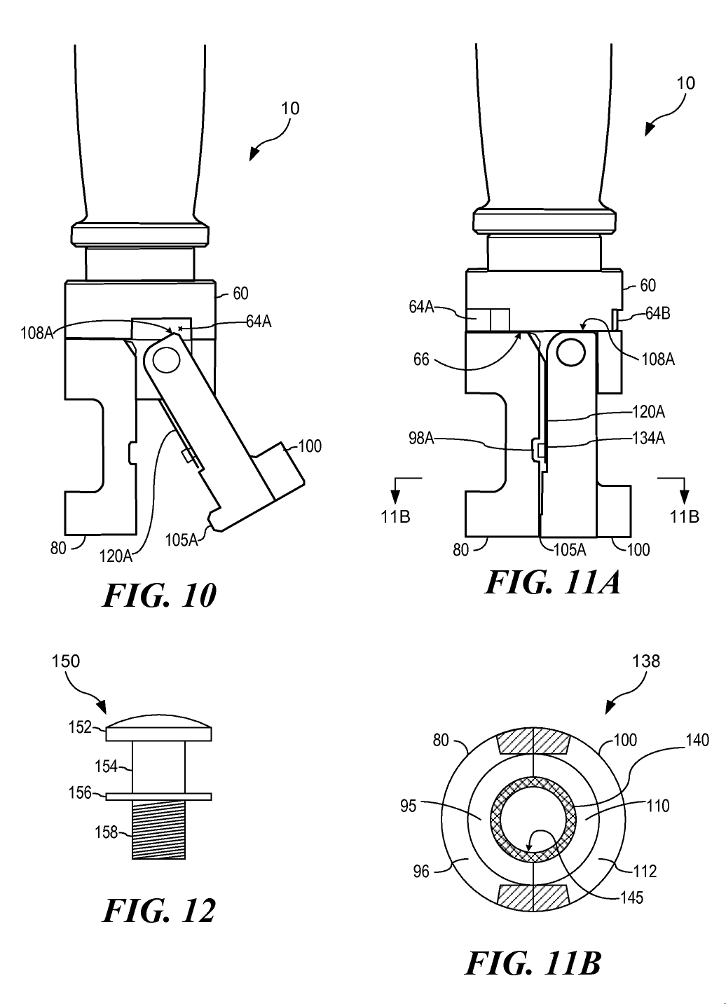

Turning now to FIG. 10, the removal tool 10 may be configured with the locking member 60 in an open position. The orientation of the locking member 60 relative to the other components may be partially secured by the engagement of the ball and spring assembly 130 (FIG. 8) with one of the detents 72A, 72B, of the limit track 70 (see FIG. 4A). In this configuration, the ‘A’ components of the operation of the clamping mechanism 20 may be detailed. The ‘B’ components may operate similarly to the ‘A’ components. With the locking member 60 in the open position, the cam recess 64A may be aligned with the cam lobe 108A of the pivotal clamp member 100. As a result, the pivotal clamp member 100 may be resiliently biased by the resilient member 120A away from the connector clamp member 80, engaging the cam lobe 108A with the corresponding cam recess 64A. The alignment protrusion 105A may be fully withdrawn from the connector clamp member 80. A tool adaptor device, detailed later, may be inserted into the clamping mechanism 20 while the clamping mechanism 20 is in this configuration.

Turning now to FIG. 1A, the removal tool 10 may be configured with the locking member 60 in a locked position. The orientation of the locking member 60 may be partially secured by the engagement of the ball and spring assembly 130 (FIG. 8) with the other one of the detents 72A, 72B, of the limit track 70 (see FIG. 4A). In this configuration, the ‘A’ components of the operation of the clamping mechanism 20 may be detailed. The ‘B’ components may operate similarly to the ‘A’ components. Prior to rotating the locking member 60 to the locked position, the pivotal clamp member 100 may be closed, or brought adjacent to the connector clamp member 80. The rotation of the pivotal clamp member 100 may disengage the cam lobe 108A from the cam recess 64A. As a result, the locking member 60 may then be rotated to the locked position. With the locking member 60 in the locked position, the cam recess 64A may not be aligned with the cam lobe 108A of the pivotal clamp member 100. Instead, the locking distal surface 66 may abut the cam lobe 108A. The abutment between the cam lobe 108A and the locking distal surface 66 may counteract the bias of the resilient member 120A and prevent or inhibit the pivotal clamp member 100 from moving away from the connector clamp member 80.

With the pivotal clamp member 100 in a closed position, the alignment protrusion 105A may be coupled with the connector clamp member 80. The coupling of the alignment protrusion 105A and the connector clamp member 80 may strengthen and support the locked clamping mechanism 20 (FIG. 1). Additionally, the fastener 134A for the mounting of the distal end of the resilient member 120A may engage the distal resilient mount recess 98A.

Turning now to FIG. 11B, the tool adaptor interface 138 may comprise the tool adaptor plate 95 of the connector clamp member 80 and the tool adaptor plate 110 of the pivotal clamp member 100. In addition, the tool adaptor interface 138 may comprise the tool adaptor wall 96 of the connector clamp member 80 and the tool adaptor wall 112 of the pivotal clamp member 100. When the pivotal clamp member 100 is in a closed and locked position, the tool adaptor plate 96 and the tool adaptor plate 110 may form a tool abutment surface 140. The tool abutment surface 140 may be surrounded by the tool adaptor wall 96 and the tool adaptor wall 112. The inner perimeter of the tool abutment surface 140 may comprise a tool accommodating circumference 145.

### Tool Adaptor Device

Turning now to FIG. 12, an example of an embodiment of a tool adaptor device 150 of the present invention may be shown. The tool adaptor device 150 may comprise a tool adaptor head 152, a tool adaptor neck 154, a tool adaptor flange 156, and a tool attachment interface 158. A distal surface of the tool adaptor head 152 may be configured to abut the tool abutment surface 140 of the tool adaptor interface 138 (see FIG. 11B) of a removal tool 10 in a locked condition (see FIG. 11A). The difference in circumference from the tool adaptor neck 154 to the tool adaptor head 152 may partially define the tool abutment surface 140.

Additionally, the tool adaptor flange 156 may abut an opposing surface of the tool adaptor interface 138, constraining the movement of the tool adaptor device 150 along a central axis relative to the tool adaptor interface 138. The tool attachment interface 158 may threadably attach to a proximal end of a seized medical instrument (not show). Although a threaded attachment is shown for this embodiment of the tool adaptor device 150, many other attachment methods may be used depending upon the particular seized medical instrument. In certain embodiments, a wide assortment of tool adaptor devices 150 may be interchangeably used with the removal tool 10 (FIG. 1) to aid in removing seized medical instruments. However, it should be understood that other instruments, in addition to medical instruments, may be removed with removal tool 10.

### Method of Removing Seized Instrument

After determining that a medical instrument may be caught in vivo, a surgeon may select the appropriate tool adaptor device 150 (see FIG. 12) corresponding to the seized instrument. The surgeon may attach the tool attachment interface 158 to a proximal end of the seized instrument. The surgeon may then obtain a removal tool 10 configured in an unlocked or open position. The tool adaptor plate 95 of the connector clamp member 80 and the tool adaptor plate 110 of the pivotal clamp member 100 (see FIG. 11B) may fit around the tool adaptor neck 154 (FIG. 12) of the selected tool adaptor device 150. The tool adaptor head 152 may abut one surface of the tool adaptor interface 138 (FIG. 11B), and the tool adaptor flange 156 may abut the other surface of the tool adaptor interface 138.

The tool adaptor device 150 may be constrained to move in an axial direction relative to the removal tool 10 due to the interaction of the tool adaptor head 152 and the tool adaptor flange 156 with the tool adaptor plates 95, 112 of the tool adaptor interface 138. The tool accommodating circumference 145 may enclose the tool adaptor neck 154. Certain embodiments of the tool adaptor interface 138 and the tool adaptor device 150 may comprise a substantially cylindrical interface between the tool adaptor neck 154 and the tool accommodating circumference 145. However, other embodiments of the tool adaptor neck 154 and the tool accommodating circumference 145 may include an interface that comprises protrusions or other shapes and forms of corresponding configurations so as to prevent or inhibit the rotation of the tool adaptor device 150 relative to the tool adaptor interface 138.

With the pivotal clamp member 100 in a locked or closed position, the locking ring 60 (FIG. 2) may be rotated to lock the pivotal clamp member 100 in position. The removal tool 10 may be securely coupled to the tool adaptor device 150 along an axis of the slide shaft 4 (FIG. 1). The surgeon may then translate the slide weight 6 (FIG. 1) along the slide shaft 4, impacting the handle 2 at one end of the slide shaft 4 and/or the retainer 40 (FIG. 2) at the other end of the slide shaft 4. The force of the impact from the slide weight 6 may be transferred along the removal tool 10 to the seized medical instrument, via the tool adaptor device 150. Repeated impacts may provide enough force to free the seized medical instrument. In addition, the attachment of the tool adaptor device 150 to the tool adaptor interface 138 and the sliding of the slide weight 6 may be configured to be performed while the surgeon maintains control over the seized medical instrument with one hand. Therefore, certain embodiments of the removal tool 10 may be configured to be manipulated with the surgeon's other hand performing the various steps of the method.

After the medical instrument is freed, the surgeon may rotate the locking ring 60 to an unlocked position. When the locking ring 60 reaches an unlocked position, the resilient members 120A, 120B (FIG. 9) may rotated the pivotal clamp member 100 to an open position, thereby releasing the tool adaptor device 150. The tool adaptor device 150 may then be removed from the medical instrument and the medical instrument used for further surgical procedures.

Although the method may be described as attaching the tool adaptor device 150 after a medical instrument becomes seized, the tool adaptor device 150 may be attached to the medical instrument prior to use. Alternatively, a proximate end of the medical instrument may comprise a structure substantially equivalent to the tool adaptor device 150, thereby enabling the removal tool 10 to be attached directly to the end of the medical instrument.

### Tool Adaptor Device

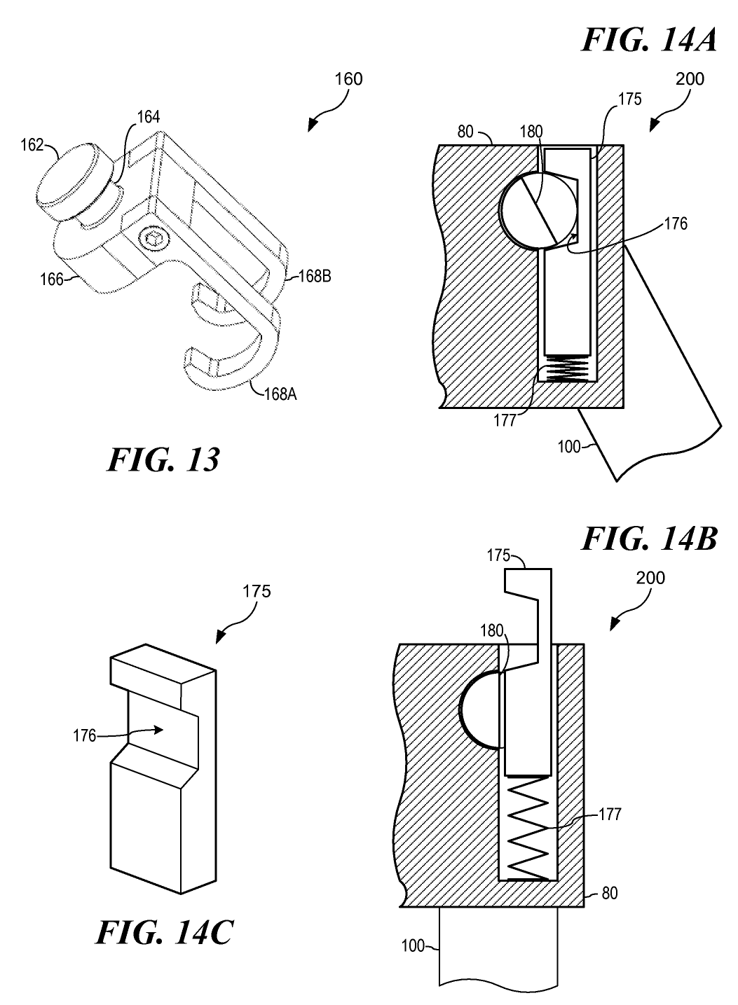

Turning now to FIG. 13, another embodiment of a tool adaptor device of the present invention may be configured as a general tool adaptor device 160. The general tool adaptor device 160 may comprise a general tool head 162, a general tool neck 164, a general tool base 166, and support hooks 168A, 168B. The general tool head 162 may abut the tool abutment surface 140 of the tool adaptor plates 95, 112 of the tool adaptor interface 138 (see FIG. 11B). The general tool base 166 may abut another surface of the tool adaptor plates 95, 112, constraining the general tool adaptor device 160 along an axis of the general tool head 162 relative to the tool adaptor interface. The general tool neck 164 may be configured to be correspond to the tool accommodating circumference 145.

The support hooks 168A, 168B may be securely attached to the general tool base 166 or integrally made with the general tool base 166. The support hooks 168A, 168B may engage a t-shaped handle of a seized medical instrument and transfer an impact force from the slide weight 6 (FIG. 1) to the seized medical instrument. Attachment of the general tool adaptor device 160 to the removal tool 10 may allow the removal tool 10 to be used with a variety of seized medical instruments.

### Locking Member

Turning to FIG. 14A another embodiment of a locking member of the present invention may comprise a locking member 175. The locking member 175 may be used in addition to or in place of the locking member 60 (FIG. 2). Another embodiment of the clamping mechanism 200 may comprise a locking member 175. The locking member 175 may comprise a rotation recess 176. The locking member 175 may be biased by a resilient member 177 in a locking direction. The pivotal clamp member 100 may be rotatably connected to the connector clamp member 80 via a cylindrical shaft 180 comprising a substantially planar section.

As the pivotal clamp member 100 of the clamping mechanism 200 may be rotated to a locked position (see FIG. 14B), the planar section of the cylindrical shaft 180 may correspondingly rotate. As the planar section of the cylindrical shaft 180 directly opposes the locking member 175, the locking member 175 may be free to move relative to the connector clamp member 80, due to the bias of the resilient member 177. The planar section of the cylindrical shaft 180 may then abut a corresponding section of the locking member 175. Consequently, the abutting of the corresponding sections may restrain or prevent further rotation of the cylindrical shaft 180 and the pivotal clamp member 100 attached to the cylindrical shaft 180.

In order to release the pivotal clamp member 100, the locking member 175 may be pressed against the bias of the resilient member 177, aligning a rotation recess 176 with the cylindrical shaft 180. The cylindrical shaft 180 may then freely rotate, thereby allowing the attached pivotal clamp member 100 to pivot to an opened position. The pivotal clamp member 100 may automatically pivot to an opened position due to the bias of a resilient member, such as the leaf spring resilient member 120 (FIG. 2) for example. Turning to FIG. 14C, an example of the configuration of the locking member 175 that comprises a rotation recess 176 may be substantially rectangular. However, any configuration providing a rotation recess and a planar section configured as an abutment surface may be use, such as for example, a pin or key.

### Locking Member

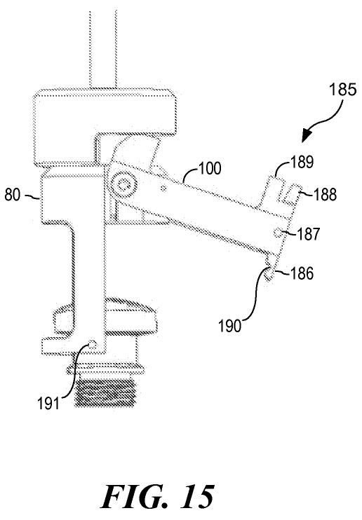

Turning now to FIG. 15, another embodiment of a locking member of the present invention may comprise a locking member 185. The locking member 185 may be used in addition to or in place of the locking member 60. The locking member 185 may comprise a latch member 186, pivot pin 187, movable handle member 188, and fixed handle member 189. The latch member 186 may be pivotally connected to a pivotal clamp member 100 via a pivot pin 187. One end of the latch member 186 opposing the connector clamp member 80 may comprise a catch 190. The other end of the latch member 186 may comprise a movable handle member 188.

The movable handle member 188 may oppose the fixed handle member 189. By forcing the movable handle member 188 toward the fixed handle member 189, the latch member 186 may be rotated about the pivot pin 187. The locking device 185 may comprise a resilient member (not shown) coupled to the latch member 186 and the pivotal clamp member 80 so as to bias the movable handle member 188 away from the fixed handle member 189. A limit (not shown) may interact with the latch member 186 to restrain the rotation of the latch member 186 to within a desired range.

The locking device 185 may comprise a pin 191 attached to the connector clamp portion 80. The pin 191 may correspond to the configuration of the catch 190 of the latch member 186. Closing the pivotal clamp member 100 may result in the latch member 186 slidably interacting with the pin 191. As the pivotal clamp member 100 becomes fully closed, the catch 190 may engage the pin 191, locking the pivotal clamp member 100 in a closed position.

To release the locking device 185, the movable handle member 188 may be moved toward the fixed handle member 189. As a result, the latch member 186 may be rotated about the pivot pin 187. Rotating the latch member 186 may disengage the catch 190 from the pin 191, releasing the pivotal clamp member 100 to pivot to an opened position.

### ALTERNATIVE EMBODIMENTS

Although a leaf spring may be shown as an example of a resilient device 120A, 120B, an embodiment of the present invention may not be limited to this example. Many alternative methods may exist for pivoting the pivotal clamp member to an open position, including, but not limited to, a coil spring intermediate to the pivotal clamp member and the connectable clamping member, a torsion spring interacting about the axis of the pivot points of the pivotal member, and natural or electromagnetic attraction and repulsion, among others.

The slide shaft 4, handle 2 and retainer 40 may be shown as separate members. However, the slide shaft 4, handle 2, and retainer 40, may be machined from a single piece of material to provide for a strengthened assembly. In such a case, the slide weight 6 may be made of two separately attachable halves and assembled around the slide shaft 4.

A resilient device 120, cam lobes 108A, 108B, and cam recesses 64A, 64B, may be used to allow the pivoting and locking of the pivotal clamp member 100 (see FIGS. 10 and 11, some components may not be visible in both views). However, a geared interface on the top of one of the pivotal support arms and a corresponding geared interface on the lower surface of the locking member may provide a mechanism to open and close the pivotal clamp member 100 relative to the rotation of the locking member. As the locking member is rotated, the gear on the locking member may engage the gear on the top of the pivotal support arm, thereby rotating the pivotal clamp member 100. In addition, other gear, linkage, or connector systems may be used.

The locking member 60 may be shown as rotating about the connectable clamping member 80. Another illustrative example of an embodiment of the present invention may further comprise a locking resilient member (e.g., such as a torsion spring) coupled to the locking member 60 and the connectable clamping member 80. The locking resilient member may bias the locking member 60 in a closed or locked direction. The bias of the locking member 60 may be less than the amount needed to overcome the bias of the resilient members 120A, 120B coupled to the connector clamp member 80 and the pivotal clamp member 100. In such a case, a surgeon may be able to maintain the pivotal clamp member 100 in an open or unlocked configuration. The locking member 60 may be held in an unlocked position by the cam lobes 108A, 108B. However, upon closing the pivotal clamp member 100, the cam lobes 108A, 108B may retract from the cam recesses 64A, 64B, thereby releasing the locking member 60 to automatically rotate to a locked position due to the bias of the locking resilient member. The clamping mechanism 20 may be coupled with a tool adaptor device 150 by closing the tool adaptor interface 138 around the tool adaptor device 150. Additionally, rotating the locking member 60 from a closed or locked position to an open position, against the bias of the locking resilient member, may automatically allow the resilient members 120A, 120B to pivot the pivotal clamp member 100 to an open position, thereby releasing any previously coupled tool adaptor device 150.

Having thus described the present invention by reference to certain of its preferred embodiments, it is noted that the embodiments disclosed are illustrative rather than limiting in nature and that a wide range of variations, modifications, changes, and substitutions are contemplated in the foregoing disclosure and, in some instances, some features of the present invention may be employed without a corresponding use of the other features. Many such variations and modifications may be considered desirable by those skilled in the art based upon a review of the foregoing description of preferred embodiments. Accordingly, it is appropriate that the appended claims be construed broadly and in a manner consistent with the scope of the invention.

1. A tool system comprising:
a slaphammer comprising:
a shaft;
an impact member coupled to the shaft;
a slide weight slidably coupled to the shaft and disposed to travel substantially parallel to an axis of the shaft; and
a lockable clamp coupled to the shaft, wherein the clamp comprises:
a fixed member coupled to the shaft;
a pivotal member pivotably coupled to the fixed member; and
a resilient member coupled to the pivotal member and disposed to bias the pivotal member to an open position,
wherein the clamp defines an accommodating circumference when the clamp is in a closed configuration; and
a plurality of tool adapters, wherein a first one of the plurality of tool adapters comprises:
a first adapter interface selected from a plurality of adapter interfaces.

2. The system of claim 1 wherein the first one of the plurality of tool adapters further comprises:
a head defining a head circumference larger than the accommodating circumference; and
a neck defining a neck circumference smaller than the accommodating circumference; and
wherein a second one of the plurality of tool adapters comprises:
a head defining approximately the head circumference; and
a neck defining approximately the neck circumference.

3. The system of claim 1 wherein a second one of the plurality of tool adapters comprises a second adapter interface selected from the plurality of adapter interfaces, and wherein the second adapter interface differs from the first adapter interface.

4. The system of claim 3 wherein the first adapter interface comprises a threaded connector and the second adapter interface comprises a hook.

5. The system of claim 1 wherein the impact member comprises a handle disposed opposite the shaft from the clamp.

6. A method for removing a medical instrument, the method comprising:
coupling a tool adaptor to a medical instrument;
coupling the tool adapter to a first clamping member;
coupling the tool adapter to a second clamping member;
locking the clamping members in place; and
receiving an impact force on an impact surface such that a portion of the impact force is transferred to the medical instrument.

7. The method of claim 6 wherein locking the clamp in a closed configuration comprises:
rotating a locking member.

8. The method of claim 6 further comprising:
opening the clamp;
decoupling the clamp from the tool adapter.

9. The method of claim 8 wherein opening the clamp comprises:
pivoting a pivotal member.

10. The method of claim 6 wherein the receiving an impact force on an impact surface further comprises positioning a weight such that a weight can impact the impact surface.

11. A surgical impact device comprising:
a shaft having a first end portion and a second end portion;
a slide weight disposed on the shaft between the first and second end portions;
a clamp coupled to the second end portion wherein the clamp comprises:
a body having a first arm;
a second arm pivotably mated to the body, the second arm having a first position wherein the first arm is generally parallel to the second arm and a second position wherein the first arm is generally spaced apart at an angle relative to the second arm.

12. The surgical impact device of claim 11 further comprising at least one resilient member positioned between the first arm and the second arm, wherein the resilient member biases the second arm in the second position.

13. The surgical impact device of claim 11 further comprising a locking member rotateably coupled to the body.

14. The surgical impact device of claim 13 wherein the locking member has an opening dimensioned to at least partially receive the second arm.

15. The surgical impact device of claim 14 further comprising the locking member having an unlocked position wherein the second arm is at least partially positioned within the opening of the locking member.

16. The surgical impact device of claim 15 further comprising the locking member having a locked position wherein the opening is adjacent to the first arm.

17. The surgical impact device of claim 13 further comprising a rotational limiting interface disposed at an interface of the locking member and the body.

18. The surgical impact device of claim 11 wherein the second arm has a first surface having one or more protrusions and the first arm has a second surface having one or more slots dimensioned to receive the one or more protrusions.

19. The surgical impact device of claim 11 wherein the first and second arms each have a generally circular outer surface and a generally circular inner surface that define a window that extends transversely through the first and second arms.

20. The surgical impact device of claim 11 further comprising an impact member coupled to the first end portion.
